(19) United States
(12) Patent Application Publication
(10) Pub. No.: US 2011/0092759 A1
(43) Pub. Date: Apr. 21, 2011

(54) MOBILE SYSTEM FOR ELECTRON BEAM INTRAOPERATIVE RADIATION THERAPY

(75) Inventors: YOURI ALEXANDROVICH KOUBYCHINE MERKULOV, BARCELONA (ES); NIKOLAY IVANOVICH PAKHOMOV, MOSCOW (RU); VASILY IVANOVICH SHVEDUNOV, MOSCOW (RU)

(73) Assignee: Universitat Politècnica de Catalunya, Barcelona (ES)

(21) Appl. No.: 12/760,753

(22) Filed: Apr. 15, 2010

Related U.S. Application Data
(63) Continuation-in-part of application No. PCT/IB2008/002791, filed on Oct. 17, 2008.

(30) Foreign Application Priority Data
Oct. 17, 2007 (ES) P200702823

(51) Int. Cl. A61N 5/00

(52) U.S. Cl. 600/1

(57) ABSTRACT

A mobile system for electron beam intraoperative radiation therapy uses a race-track microtron placed in a housing playing the role of the vacuum chamber, which is supported by a positioner providing its motion with six degrees of freedom with respect to a patient. The positioner is a part of a mobile mechanical structure which houses an ion pump, a microwave source, waveguide elements, a modulator with a pulse transformer and a cooler. The intraoperative radiation therapy system has a tube-like unit which couples the vacuum chamber with the mobile supporting mechanical structure and provides three functions, namely pumping out of the air from the vacuum chamber, feeding of the race-track microtron accelerating structure with radiofrequency power and rotation of the vacuum chamber with respect to the horizontal axis of the unit.

### CROSS-REFERENCE TO COPENDING PATENT APPLICATIONS

The present application is a continuation-in-part of PCT/ES2008/000643 filed Oct. 16, 2008, which in turn claims the benefit of priority from Spanish patent application Ser. No. P200702724 filed Oct. 17, 2007.

### BACKGROUND OF THE INVENTION

1. Field of the Disclosure

The invention relates generally to a mobile system for electron beam intraoperative radiation therapy with a race-track microtron as the electron beam source.

2. Background Art

The Intaroperative Radiation Therapy (IORT) is a rapidly developing technique that has attracted increasing interest in modern oncology. IORT can be defined as a radiotherapy treatment technique consisting in the administration, during a surgical intervention, of a single and high radiation dose in the range of 10 Gy to 20 Gy directly to the tumor bed/environment in a surgically defined area using electron beams of energies in the range of 4 MeV to 20 MeV. This treatment method permits to avoid or to maximally reduce damaging of healthy tissues. Another important feature is that in this way it is possible to sterilize the surgery zone where some microscopic residues may remain which cannot be surgically removed and which can give rise to local relapses.

The IORT has been shown to be effective in the treatment of breast cancer, soft tissues sarcomas, gynecological, colorectal and pancreatic cancers, etc. The forms in which the IORT can be applied include the irradiation of a tumor bed after full surgical removal, irradiation of tumor residuals after a partial surgical extraction or irradiation of surgically inoperable tumors.

X-rays are not suitable for the IORT because of their high penetration power, high bone absorption and rather slow decrease of the delivered dose with the penetration depth, the feature which makes it hard to avoid affecting zones which must not be irradiated. In addition, treatments with X-ray would have long treatment times.

The penetration depth of an electron beam is precisely controlled by changing its energy, therefore IORT treatments with electrons allow to irradiate the desired zone only without damaging neighboring tissues. In addition, in this case the irradiation field can be easily shaped using external applicators.

One option would be to use “conventional”, i.e. designed for external radiotherapy (ERT), linear accelerators (linacs) for the IORT. However, this approach has several drawbacks. First of all the ERT machines do not fully satisfy criteria for the IORT. Because of their large size and weight they cannot be positioned properly for the IORT irradiation, therefore the patient must be moved that implies quite complex logistics. Also, the existing ERT linacs generate intense radiation during their operation, therefore they have to be placed in a special bunker.

As a consequence, the implementation of the IORT with linacs designed for the ERT follows one of the two schemes: (1) organization of an operation room inside the accelerator bunker, or (2) transportation of the patient, under anesthesia, from the operation room to the linac bunker and back to the operation room after the irradiation.

Both schemes have serious drawbacks. The first scheme requires a large capital outlay for the medical centre. In this case the accelerator will be used with the frequency determined by the surgical operations, that is, typically, one-three patient per day depending on the type of the tumor. As a result, the expensive machine capable of treating a high number of patients will be used with very low efficiency.

The main drawback of the scheme with patient transportation from the operation room to the accelerator bunker during the surgical operation is the increased complexity of the treatment due to risk of infection, special anesthesia requirements and more complicated logistics.

All these difficulties were the main reason why the IORT, despite its theoretical advantages, did not gain wide application till the mid-nineties of the 20th century. It was clear that a solution would be to use mobile electron beam accelerators that can easily be transported and employed directly in the operation room. With the introduction of facilities of this type in clinics at the beginning of 2000 a new era of IORT has started.

Presently, the only IORT dedicated accelerators are specially designed X-band (3 cm wavelength) and S-band (10 cm wavelength) linacs, for example Mobetron (Intarop Medical Corporation, USA) or Novac-7 (Hitesys, Italy).

The IORT dedicated facilities based on linacs have certain drawbacks. The first of them is that in order to assure the required precision of the exit beam energy a procedure of beam calibration has to be carried out before each operation. This increases the radiation load in the operation room.

Moreover, linacs do not have a simple and reliable system of changing the exit beam energy just before the irradiation in accordance of the radiotherapist decision.

A further drawback is related to the efficiency. The dose rate in the range 10-20 Gy/min necessary for the IORT is provided by the average beam current of only ˜0.2 μA. For such low current 99.9% of the RF power is just dissipated in the linac walls.

One more drawback is the following. To avoid generation of an uncontrollable current, so called dark current, the linac accelerating gradient must be below 10-15 MeV/m, hence the length of its accelerating unit only must be about 1 m. This makes the IORT facility to be rather bulky and heavy.

There exist a few patent documents related to previous proposals in the same technical field of the present invention. Thus there can be cited the United States patents U.S. Pat. No. 5,321,271 “Intraoperative electron beam therapy system and facility” and U.S. Pat. No. 5,635,721 “Apparatus for the linear acceleration of electrons, particularly for intraoperative radiation therapy”, as well as the patent application publication No.: US-A.-2005/0259786 “Machine for Intraoperative radiation therapy”. All these patents refer to intraoperative radiation therapy facilities in which the beam of electrons is generated by a linear accelerator.

In the articles “Equipo para radioterapia intraoperatoria basado en un microtrón de pista de 12 MeV” published in the journal “Fisica Médica”, Vol. 8, No. 1 (2007), “Conceptual design of the miniature electron accelerator dedicated to IORT” published in “Proceedings of RuPAC XIX”, Dubna 2004, and “Design of 12 MeV RTM for multiple applications” published in “Proceedings of the 10th European Particle Accelerator Conference EPAC-2006” (Edinburgh, June 26-30, 2006), p. 2340-2342 (2006), written by the authors of the present invention in collaboration with other specialists, a description of a mobile system for electron beam intraoperative radiation therapy comprising in a race-track microtron as accelerator of electrons is described. The microtron is placed inside a vacuum chamber attached to a mobile supporting structure which provides the accelerator positioning with six degrees of freedom with respect to the patient.

In the quoted articles some general features of an IORT dedicated mobile system using a race-track microtron are described. The results and conclusions exposed there are based on calculations and numerical simulations of the theoretical design and do not give details of concrete technical solutions required for building the microtron components.

### SUMMARY OF THE INVENTION

Taking into account the state of the art inventors have found necessary to provide an alternative IORT dedicated mobile system in which the electron beam with improved characteristics is generated by a compact electron accelerator of race-track microtron type. Such system has certain advantages with respect to existing devices, namely a lower weight, simplicity in operation, smaller dimensions of the accelerator head, and also more compact and practical distribution of the facility components.

As a realization of such proposal the present invention describes a mobile system for the electron beam intraoperative radiation therapy, whose general features were outlined in the articles mentioned above. The system comprises a race-track microtron as the electron accelerator generating the beam of electrons, the microtron is placed inside a chamber in which high vacuum is created and which is joined to a mobile supporting mechanical structure which provides the positioning of the accelerator with respect to the patient with six degrees of freedom. The race-track microtron is fed by a radiofrequency source with a radiofrequency power through a system of electromagnetic wave transportation.

The characteristic features of the system of the proposed invention is that the pumping out of the chamber to create high vacuum in the said chamber and the supply of the radiofrequency electromagnetic wave are realized through the same unit which joins the said chamber with the said mechanical supporting structure. The unit provides also the rotation of the said chamber with respect to the horizontal axis thus achieving the practical and compact design of the facility mentioned above.

In a preferred embodiment of the IORT system of the present invention the electron race-track microtron is placed in a chamber which forms the facility accelerator head. The chamber is joined to a module which houses a vacuum pump. The chamber and the module are moved and positioned by a robotic arm. Elements of the radiofrequency system, modulator, power supply source and cooling system are placed in a supporting structure. The reduced dimensions of the accelerator head are due to the use of a C-band accelerating structure and end magnets with a rare earth permanent magnet material as a source of magnetic field.

### BRIEF DESCRIPTION OF THE DRAWINGS

The abovementioned main features of the invention can be understood from the drawings of the preferred embodiment shown in attached figures, which should be considered merely as illustrations.

FIG. 1 is a schematic representation of the preferred embodiment of the electron beam source with the following elements indicated: 1 electron gun; 2 accelerating structure; 3 and 4 end magnets; 5 focusing quadrupole; 6 extraction magnets; 7 chamber; 8 output beam.

FIG. 2 shows the end magnet of the electron beam source of FIG. 1; the upper drawing shows the view from above, the bottom drawing shows the transverse cross section of the end magnet along the A-A plane.

FIG. 3 is a block diagram of the preferred embodiment of main components of the IORT system of the invention and interconnections between them.

### DETAILED DESCRIPTION OF THE PREFERRED EMBODIMENTS

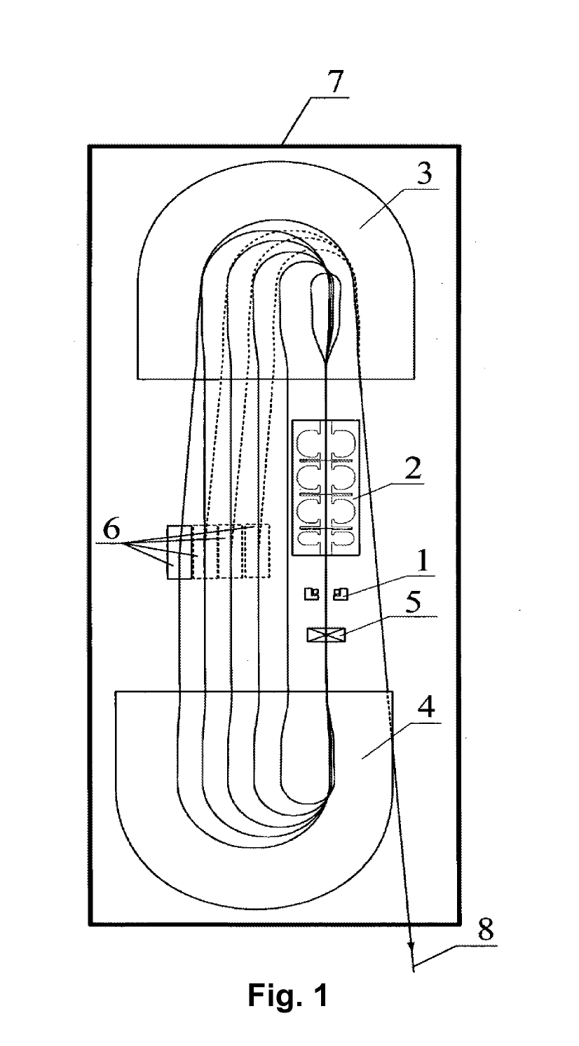

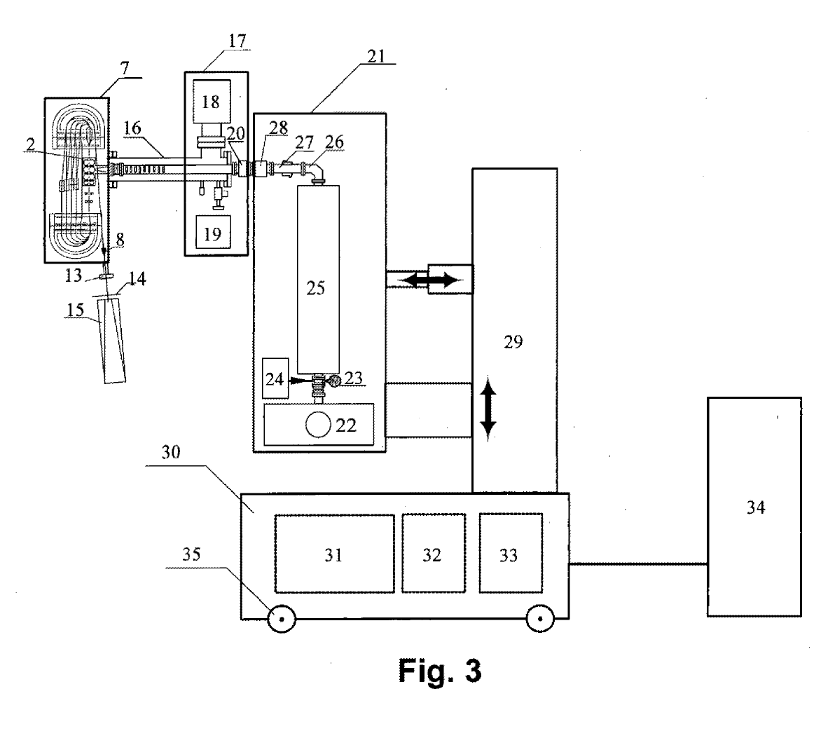

The content of the present invention is a mobile system of electron beam intraoperative radiation therapy (IORT), shown schematically in FIG. 3, which includes a race-track microtron (shown in more detail in FIG. 1) as an accelerator of electrons with accelerating structure 2 placed inside chamber 7, where a vacuum is maintained, coupled to a mobile supporting mechanical structure which provides the positioning of said race-track microtron with respect to the patient with six degrees of freedom, said microtron is fed by source 22 of radiofrequency (RF) electromagnetic wave and modulator 31.

The present invention is characterized by unit 16, which couples said chamber 7 with said mobile supporting mechanical structure. This same unit 16 provides pumping out of the air to obtain the vacuum in chamber 7, the supply of the RF power to said accelerating structure 2 of said microtron and rotation of said chamber 7 with respect to the horizontal axis. Said unit 16 includes at least one tube or a tubular structure which provides these functions.

In the preferred embodiment illustrated in FIG. 3 unit 16 consists of a tube for pumping out of the air and a waveguide placed inside this tube. One end of this tube is joined to said chamber 7 housing the race-track microtron and its other end is joined to module 17 which forms part of said mobile supporting mechanical structure and which houses vacuum pump 18.

An IORT dedicated system, which is supposed to operate in a standard hospital operation room, should satisfy the following requirements:
(1) The electron beam source should provide an electron beam of the energy variable in the range from 4 to 12 MeV, for a given energy the beam must have low energy spread.
(2) The system must possess means to position the electron beam source with high precision, therefore the electron beam source must be of small enough size and weight.
(3) The system must be mobile, so that it can be moved while being in the operation mode within the operation room, of small enough dimensions which permit its easy displacements within a standard operation room and its transportation within the hospital, and of sufficiently low weight so that it can be placed in a standard operation room without any floor reinforcement.
(4) The IORT system must be equipped with adequate means of shielding for both the radiation generated inside the system itself and for the scattered radiation, so that it can be used in a standard operation room without additional shielding of this or adjacent rooms.

The preferred embodiment of this invention, which comprises the IORT system with characteristics described below and a race-track microtron as the electron beam source, meets these requirements.

The race-track microtron (RTM) is a known type of particle accelerator which essentially consists of two 180°-degree bending magnets, often called end magnets, a linear accelerating structure situated between them, a system of injection of electrons and a system of extraction of the accelerated beam. The injected electrons are accelerated by the accelerating structure and are directed towards one of the end magnets. The constant magnetic field generated between the poles of the magnet forces the electrons to make a 180°-turn directing them to the second end magnet. After making a 180°-turn in the second magnet the electrons return to the accelerating structure to gain a further energy increase. In this way the electron beam makes a few recirculations along consecutive orbits with increasing bending radius inside the end magnets and with their common part passing through the accelerating structure. The beam circulates inside a vacuum chamber of corresponding geometry. The accelerating structure is fed by a radiofrequency (RF) source. The beam is focused by certain profile of the magnetic field in the end magnets and by magnetic quadrupole lenses placed at certain positions on the beam trajectory. Once the beam gains the required energy its trajectory is deviated by an extraction magnet. The extracted beam passes through an exit window, a sequence of devices, like diffusion foils or applicators, which shape the irradiation field towards the operation bed to be irradiated.

As an electron beam source for an IORT dedicated system an RTM has certain advantages with respect to linear accelerators. First of all, for a given energy gain and accelerating field gradient the accelerating structure of an RTM with N recirculations has N times shorter length and N times lower weight than those of the linear accelerator.

A second advantage is that since the final beam energy is gained in N beam passages through the accelerating structure and since the RF power consumption by the beam is negligible the required RF power is reduced by a factor of N with respect to a corresponding linear accelerator. As a consequence, the RF source and modulator voltage, power, cost and system dimensions and weight are essentially reduced.

A further advantage is that in the RTM the beam can be extracted from any orbit, thus allowing to change the beam energy with a fixed step in a wide range.

Finally, since the RTM end magnets act as a precise spectrometer no special beam energy control is necessary and, in addition, the energy spread of the exit RTM beam is only ˜50-100 keV and its spectrum does not have low energy tail.

The preferred embodiment of the electron beam source of the invention is an RTM with the energy gain per turn equal to approximately 2 MeV, exit beam energies 6, 8, 10 and 12 MeV, average beam current regulated by pulse repetition rate from several tens of nA to several tens of μA and nominal delivered dose rate of 10-30 Gy/min. As shown in FIG. 1 the RTM comprises accelerator head which includes an on-axis electron gun 1, a C-band accelerating structure 2 situated between end magnets 3 and 4, a quadrupole magnet 5 acting as a magnetic lens and extraction magnets 6 placed inside chamber 7. Each of the orbits with beam energy 6, 8, 10 or 12 MeV has its own extraction magnet which is placed at the axis of the orbit corresponding to the energy of exit beam 8.

Said end magnets 3, 4 said accelerating structure 2, said electron gun 1 and said quadrupole magnet 5 are fixed on a common platform inside chamber 7.

A C-band standing wave accelerating structure 2 comprised by a series of cavities is optimal in fulfilling various criteria. Thus, on one hand, the wavelength is short enough for the sizes of the accelerating structure and the end magnets to be sufficiently small and their weights to be sufficiently low. On the other hand, the wavelength is long enough for the capture efficiency of injected non-relativistic particles to be sufficient even with one accelerating cavity of length shorter then the half-wavelength and for the distance between successive orbits to be sufficiently large for placing the extraction magnets.

For the preferred embodiment of this invention a low energy injection scheme with on-axis electron gun 1 is implemented. Therefore there is no need neither in separate pre-accelerator of the injected beam, as in the case of high energy injection, nor additional magnets, deflectors, etc. needed in the case of schemes with an off-axis electron gun.

In the preferred embodiment of the invention the magnetic field in end magnets 3 and 4, in quadrupole focusing magnetic lens 5 and extraction magnets 6 is generated by a permanent magnet material, preferably a rare-earth permanent magnet (REPM) material.

Magnetic systems based on REPM materials have certain advantages with respect to those based on electromagnets. First of all, a magnetic system with REPM has no coils, therefore it does not require power supply and cooling and can be placed inside the vacuum chamber. A second advantage is that an REPM material allows to get a strong enough magnetic field (up to 1.8 T) in a small volume and to build, for a required range of energies, a more compact and less heavy magnetic system as compared to electromagnets. Finally, the accelerator operation is considerably simpler and the reproducibility of characteristics of the magnetic system is higher in the case of the REPM material magnets.

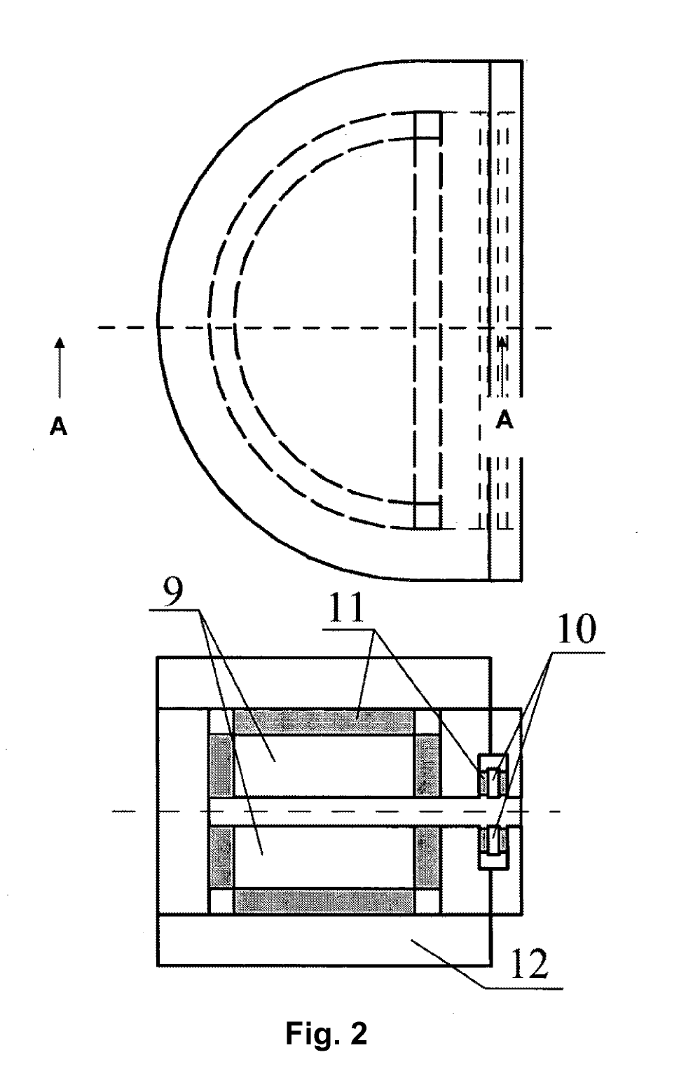

In the preferred embodiment of the invention end magnets 3 and 4 is of the box-type design as it is shown in FIG. 2. Each end magnet consists of main pole 9, reverse pole 10 and REPM material 11 surrounded by yoke 12.

The design of magnets 3, 4, 5 and 6 using the REPM material as the source of the magnetic field allows to reduce the size and weight of the accelerator head and place all the elements of the electron beam source in the vacuum, so that chamber 7 plays the role of the vacuum chamber where high vacuum is maintained.

In the preferred embodiment of this invention extracted beam 8 passes through window 13, as it is shown in FIG. 3, at the beam outlet which keeps high vacuum inside chamber 7, and follows an exit transport line. This line includes scattering foil 14 and applicator tube 15 which shape the irradiation field required for the IORT treatment.

As shown in FIG. 3 in the preferred embodiment of this invention chamber 7 is connected via tube 16 to module 17 which houses vacuum pump 18, electron gun high voltage transformer 19 and vacuum window 20. This module is coupled through a rotary joint to module 21 which houses RF source 22, pressure unit 23 with isolating gas filling system 24, circulator 25 with dummy loads, waveguide H-bend 26, double directional coupler 27 and RF rotary joint 28. Modules 17, 21 and 29 are parts of a robotic arm which supports, rotates and positions RTM chamber 7. The robotic arm is mounted on base unit 30 which also houses modulator 31, cooler 32 and power supplies 33. The operation of the IORT system is controlled by control system 34 which is placed in a separate module.

By means of unit 16 the air extraction for creating and maintaining vacuum in chamber 7, feeding accelerating structure 2 with RF power and rotation of chamber 7 with respect to the horizontal axes are achieved.

The RF power feeding accelerating structure 2 is generated by magnetron 22 such as Communications & Power Industries model SFD-313-V, which is capable of operating at a peak power 1 Megawatts at a duty cycle of 0.001. The preferred magnetron is mechanically tunable and generates pulses of length 2 microseconds.

In the preferred embodiment the IORT system is equipped with modulator 31 such as ScandiNova Systems AB model M1 which is capable of producing pulses of peak voltage 36 kV with a width at top of 3 microseconds, voltage flatness ±1.0% and maximal duty cycle 0.001.

The robotic arm allows to position RTM chamber 7 with sufficient precision in such a way that the outlet of applicator 15 is placed in a required point and at a required angle with respect to the patient so that the operation bed can be irradiated in a most adequate way. For this purpose the robotic arm provides three degrees of freedom of motion of RTM chamber 7, namely translations in the vertical direction and rotations with respect to the two mutually orthogonal horizontal axes. Three more degrees of freedom, namely the motion in the two horizontal directions and rotation around the vertical axis, are provided by the four motorized wheels 35 placed beneath base unit 30.

In the preferred embodiment of the invention the radiation created by the RTM is due to parasitic electron beam losses inside chamber 7 and at the beam outlet window. Without special measure taken it would be produced mainly at higher energy orbits. To reduce significantly the beam losses at high energy orbits and make them practically negligible a narrow aluminium collimator is installed at the 4 MeV orbit which cuts out a part of the beam which is too much off-axis or off-momentum. With additional lead collimator placed at the 4 MeV orbit the radiation generated by the RTM is reduced to the acceptably low level so that no additional shielding of the accelerator is needed. The radiation generated as a result of interaction of the beam with the patient tissues is strongly collimated in the forward direction with the aperture angle about 30°. To reduce the dose produced by this radiation to a safe level a lead beam stopper of thickness about 8 cm is placed beneath the patient. The required dimensions of the beam stopper depend on maximal irradiation field size and the separation between the plate and the patient.

In principle, the specific characteristics of the preferred embodiment of the invention allow to get an IORT system easier in operation, of lower weight, smaller dimensions of the accelerator head and with a better distribution of system components than the existing IORT systems.

The invention described here may be modified or adapted to other applications by those skilled in the art who can introduce changes and modifications in the preferred embodiment described above without going beyond the scope of the invention as defined in the claims.

What is claimed is:

1. A mobile electron beam intraoperative radiation therapy system comprising a race-track microtron as a source of accelerated electrons, said race-track microtron comprising an accelerating structure placed inside chamber in which vacuum is maintained, said chamber being coupled to a mobile supporting mechanical structure, comprised by said mobile electron beam intraoperative radiation therapy system, and apt to position said race-track microtron with respect to the patient with six degrees of freedom, a radiofrequency source and a modulator feeding said race-track microtron,
wherein said chamber is coupled to said mobile supporting mechanical structure by a single unit providing three functions:
pumping out of the air from said chamber,
feeding said accelerating structure of said race-track microtron with RF power,
displacement or rotation of said chamber,
said single unit comprising at least one tube or a tubular structure for pumping out of the air and a waveguide placed inside said tube or tubular structure for the electromagnetic wave transport of said RF power feeding.

2. The system of claim 1, wherein said mobile supporting mechanical structure comprises modules housing elements of said race-track microtron and said electron beam intraoperative radiation therapy system.

3. The system of claim 2, wherein one end of said unit is joined to said chamber which houses the race-track microtron and its other end is joined to a first module which forms part of said mobile supporting mechanical structure and which houses a vacuum pump.

4. The system of claim 1, wherein said race-track microtron comprises:
two 180° bending magnets and one particle accelerating structure placed between said magnets;
one on-axis electron gun and one focusing quadrupole placed on the axis of said particle accelerating structure, and
one set of beam extraction magnets which are remotely placed at one of the said race-track microtron orbits from which electron beam is extracted,
said 180° bending magnets, said accelerating structure, said electron gun and said focusing quadrupole being fixed on a common platform, said platform is placed inside a vacuum box.

5. The system of claim 3, wherein said first module also houses a high voltage transformer for power supply to the cathode of an electron gun of said race-track microtron.

6. The system of claim 3, wherein said first module is coupled, by means of a rotary joint, to a second module which forms part of said mobile supporting mechanical structure and which houses a RF source, a gauge and elements for guiding and conditioning the RF electromagnetic wave, said second module being joined to a robotic system supporting structure which displaces and rotates chamber and modules.

7. The race-track microtron of claim 4, wherein the magnetic field in said 180° bending magnets, said focusing quadrupole and said extraction magnets is generated by a rare-earth permanent magnet material as a field source.

8. The race-track microtron of claim 4, wherein said particle accelerating structure comprises a chain of coupled RF cavities and operates in C-band.
